(19) United States
(12) Patent Application Publication
(10) Pub. No.: US 2004/0063759 A1
(43) Pub. Date: Apr. 1, 2004

(54) Novel piperidine derivatives

(75) Inventors: Laura C. Blumberg, Waterford, CT (US); Matthew F. Brown, Stonington, CT (US); Matthew M. Hayward, Old Lyme, CT (US); Christopher S. Poss, Baltic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/616,844

(22) Filed: Jul. 8, 2003

Related U.S. Application Data
(60) Provisional application No. 60/397,108, filed on Jul. 18, 2002.

(51) Int. Cl. A61K 31/445; C07D 211/06

(52) U.S. Cl. 514/317; 546/226; 546/217; 546/223

(57) ABSTRACT

A compound of the formula

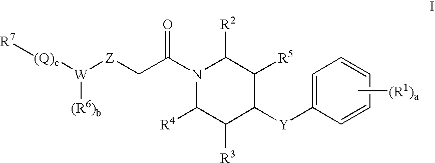

wherein a, b, c R¹, R², R³, R⁴, R⁵, R⁶, R⁷, Q, W, Y, and Z are defined as above, useful as potent and selective inhibitors of MIP-1α (CCL3) binding to its receptor CCR1 found on inflammatory and immunomodulatory cells (preferably leukocytes and lymphocytes).

This application claims the benefit of priority of U.S. provisional Patent Application Serial No. 60/397,108 filed Jul. 18, 2002, which is incorporated herein in its entirety for all purposes.

### BACKGROUND OF THE INVENTION

The present invention relates to novel piperidine derivatives, methods of use and pharmaceutical compositions containing them.

The compounds of the invention are potent and selective inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment or prevention of autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. Compounds of this invention are also potentially useful for the treatment or prevention of cancer metastasis, including but not limited to breast cancer. Compounds of this invention may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Compounds of this invention may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

MIP-1α and RANTES are soluble chemotactic peptides (chemokines) which are produced by inflammatory cells, in particular CD8+ lymphocytes, polymorphonuclear leukocytes (PMNs) and macrophages, J.Biol. Chem., 270 (30) 29671-29675 (1995). These chemokines act by inducing the migration and activation of key inflammatory and immunomodulatory cells. Elevated levels of chemokines have been found in the synovial fluid of rheumatoid arthritis patients, chronic and acute rejecting tissue from transplant patients and in the nasal secretions of allergic rhinitis patients following allergen exposure (Teran, et al., J. Immunol., 1806-1812 (1996), and Kuna et al., J. Allergy Clin. Immunol. 321 (1994)). Antibodies which interfere with the chemokine/receptor interaction by neutralizing MIP1α or gene disruption have provided direct evidence for the role of MIP-1α and RANTES in disease by limiting the recruitment of monocytes and CD8+ lymphocytes (Smith et al., J. Immunol, 153, 4704 (1994) and Cook et al., Science, 269, 1583 (1995)). Together this data demonstrates that CCR1 receptor antagonists would potentially be an effective treatment of several immune based diseases. The compounds described within are potent and selective antagonists of the CCR1 receptor.

### SUMMARY OF THE INVENTION

The present invention relates to a compound of the formula

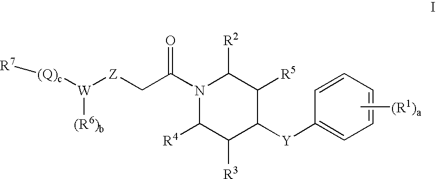

or pharmaceutically acceptable salts, tautomers, and pro-drugs thereof; wherein

a is 1, 2, 3, 4 or 5;

b is 0, 1, 2, 3, or 4;

c is 0 or 1;

Q is (C₁-C₆)alkyl;

W is (C₆-C₁₀)aryl or (C₂-C₉)heteroaryl;

Y is oxygen, or NR⁸ wherein R⁸ is hydrogen or (C₁-C₆)alkyl;

Z is oxygen or NR⁹, where R⁹ is hydrogen, (C₁-C₆)alkyl, or acetyl;

each R¹ is independently selected from the group consisting of: hydrogen, halo, cyano, nitro, trifluoromethyl, trifluoromethoxy, (C₁-C₆)alkyl, hydroxy or (C₁-C₆)alkylcarbonyloxy, (C₁-C₆)alkoxy;

R², R³, R⁴ and R⁵ are each independently hydrogen or (C₁-C₆)alkyl optionally substituted with 1 to 3 halo groups; with the provision that at least one of R², R³, R⁴ and R⁵ is (C₁-C₆)alkyl.

each R⁶ is independentiy selected from a list consisting of: hydrogen, halo, (C₁-C₆)alkyl optionally substituted with 1 to 3 halo groups; cyano, (C₁-C₆)alkoxy, aminocarbonyl, carboxy, (C₁-C₆)alkylcarbonyl, or (C₁-C₆)alkoxy optionally substituted by 1 to 3 halo groups; and

R⁷ is selected from a list consisting of hydrogen, halo, (C₁-C₆)alkyl optionally substituted with 1 to 3 halo groups, [(C₁-C₆)alkyl]₂amino(C₁-C₆)alkylaminocarbonyl, amino(C₁-C₆)alkylaminocarbonyl, (C₁-C₆)alkylamino(C₁-C₆)alkylaminocarbonyl cyano, (C₁-C₆)alkoxy, aminocarbonyl, (C₁-C₆)alkylaminocarbonyl, [(C₁-C₆)alkyl]₂aminocarbonyl, (C₁-C₆)alkylsulfonylamino, (C₁-C₆)alkylsulfonylaminocarbonyl, ureido, aminosulfonyl, [(C₁-C₆)alkyl]₂aminosulfonyl, (C₁-C₆)alkylaminosulfonyl, [(C₁-C₆)alkyl]₂aminocarbonyl(C₁-C₆)alkylaminocarbonyl, (C₁-C₆)alkylaminocarbonyl(C₁-C₆)alkylaminocarbonyl, aminocarbonyl(C₁-C₆)alkylaminocarbonyl, (C₁-C₆)alkylsulfonylamino, hydroxy(C₁-C₆)alkylcarbonylamino, ureido(C₁-C₆)alkylaminocarbonyl, [(C₁-C₆)alkyl]₂ureido(C₁-C₆)alkylaminocarbonyl, (C₁-C₆)alkylureido(C₁-C₆)alkylaminocarbonyl, (C₂-C₉)heteroarylaminocarbonyl, carboxy, (C₁-C₆)alkoxy(C₁-C₆)alkyl(C₂-C₉)heterocyclecarbonyl, (C₂-C₉)heterocyclecarbonyl, hydroxy(C₂-C₉)heterocyclecarbonyl, aminocarbonyl(C₂-C₉)heterocyclecarbonyl, carboxy(C₂-C₉)heterocyclecarbonyl, amino(C₂-C₉)heteroaryl(C₁-C₆)alkyl, (C₁-C₆)alkylamino(C₂-C₉)heteroaryl(C₁-C₆)alkyl, [(C₁-C₆)alkyl]₂amino(C₂-C₉)heteroaryl(C₁-C₆)alkyl, (C₂-C₉)heteroarylamino(C₁-C₆)alkyl, (C₂-C₉)heteroarylaminocarbonyl(C₁-C₆)alkoxy, (C₁-C₆)alkylsulfonylaminocarbonyl(C₁-C₆)alkoxy, aminocarbonyl(C₁-C₆)alkoxy, carboxy(C₁-C₆)alkoxy, aminosulfonyl, (C₁-C₆)alkylcarbonylaminosulfonyl, hydroxy(C₁-C₆)alkylcarbonylaminosulfonyl, (C₁-C₆)alkoxycarbonylaminosulfonyl, (C₁-C₆)alkoxy(C₁-C₆)alkylcarbonylaminosulfonyl, hydroxysulfonyl, hydroxy, hydroxy(C₁-C₆)alkylaminocarbonyl, carboxy(C₂-C₉)heterocycloxy or [carboxy][amino](C₁-C₆)alkoxy, aminocarbonyl(C₁-C₆)alkylcarbonylamino, (C₁-C₆)alkylaminocarbonyl(C₁-C₆)alkylcarbonylamino, [(C₁-C₆)alkyl]₂aminocarbonyl(C₁-C₆)alkylcarbonylamino, amino(C₁-C₆)alkylcarbonylamino, (C₁-C₆)alkylamino(C₁-C₆)alkylcarbonylamino, [(C₁-C₆)alkyl]₂amino(C₁-C₆)alkylcarbonylamino, ureido(C₁-C₆)alkylcarbonylamino, (C₁-C₆)alkylureido(C₁-C₆)alkylcarbonylamino, [(C₁-C₆)alkyl]₂ureido(C₁-C₆)alkylcarbonylamino, amino(C₁-C₆)alkylsulfonylamino, amino(C₁-C₆)alkylcarbonylaminosulfonyl, (C₁-C₆)alkylamino(C₁-C₆)alkylcarbonylaminosulfonyl, [(C₁-C₆)alkyl]₂amino(C₁-C₆)alkylcarbonylaminosulfonyl, aminosulfonylamino, (C₁-C₆)alkylaminosulfonylamino, [(C₁-C₆)alkyl]₂aminosulfonylamino, (C₂-C₉)heterocycloxy, (C₂-Cg)heteroaryloxy, (C₂-C₉)heterocycleamino, (C₂-C₉)heteroarylamino, amino(C₁-C₆)alkoxy, (C₁-C₆)alkylamino(C₁-C₆)alkoxy, [(C₁-C₆)alkyl]₂amino(C₁-C₆)alkoxy, amino(C₁-C₆)alkylamino, (C₁-C₆)alkylcarbonylamino(C₁-C₆)alkylamino, ureido(C₁-C₆)alkylamino, hydroxy(C₁-C₆)alkylamino, (C₁-C₆)alkoxy(C₁-C₆)alkylamino, and (C₁-C₆)alkylsulfonylamino(C₁-C₆)alkylamino.

Preferred compounds of the formula I include those wherein R₁ is halo and a is 1 or 2.

Preferred compounds of the formula I include those wherein Y is oxygen.

Preferred compounds of the formula I include those wherein Z is oxygen.

Preferred compounds of the formula I include those wherein Z is NH.

Preferred compounds of the formula I include those wherein W is phenyl.

Preferred compounds of the formula I include those wherein W is pyridyl.

Preferred compounds of the formula I include those wherein b is 0, 1 or 2, and R⁶ is selected from a list consisting of halo, (C₁-C₆)alkyl, cyano, or (C₁-₆)alkylcarbonyl.

Preferred compounds of the formula I include those wherein c is 0, and R⁷ is selected from a list consisting of aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy.

Preferred compounds of the formula I include those wherein c is 1, and R⁷ is selected from a list consisting of (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, or carboxy.

Preferred compounds of the formula I include those wherein R² and R³ are both methyl groups and R⁴ and R⁵ are both hydrogen.

Preferred compounds of the formula I include those wherein R² and R³ are trans and Y and R³ are trans; having relative stereochemistry as shown below.

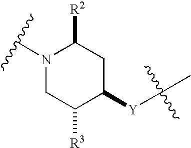

Preferred compounds of the formula I include those wherein R₁ is halo; a is 1 or 2; Y is oxygen; Z is oxygen; R² and R³ are methyl; R⁴ and R⁵ are hydrogen; R² and R³ are trans; Y and R³ are trans; W is phenyl; b is 0, 1 or 2; R⁶ is selected from the group consisting of halo, (C₁-C₆)alkyl, cyano, or (C₁-C₆)alkylcarbonyl; c is 0; and R⁷ is selected from the group consisting of: aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy.

Preferred compounds of the formula I include those wherein R₁ halo; a is 1 or 2; Y is oxygen; Z is oxygen or NH, R² and R³ are methyl; R⁴ and R⁵ are hydrogen; R² and R³ are trans; Y and R³ are trans; W is pyridyl; b is 0, 1 or 2; R⁶ is selected from the group consisting of halo, (C₁-C₆)alkyl, cyano, or (C₁-C₆)alkylcarbonyl; c is 0; and R⁷ is selected from the group consisting of: aminocarbonyl, (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, amino(C₂-C₉)heteroaryl, (C₂-C₉)heteroarylamino, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, or carboxy(C₁-C₆)alkoxy.

Preferred compounds of the formula I include those wherein R¹ is halo; a is 1 or 2; Y is oxygen; Z is oxygen; R² and R³ are methyl; R4 and R⁵ are hydrogen; R² and R³ are trans; Y and R³ are trans; W is phenyl; b is 0, 1 or 2; R⁶ is selected from the group consisting of: halo, (C₁-C₆)alkyl, cyano, or (C₁-C₆)alkylcarbonyl; c is 1; and R⁷ is selected from the group consisting of: (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, aminosulfonyl, or carboxy.

Preferred compounds of the formula I include those wherein R₁ halo; a is 1 or 2; Y is oxygen; Z is oxygen or NH, R² and R³ are methyl; R⁴ and R⁵ are hydrogen; R² and R³ are trans; Y and R³ are trans; W is pyridyl; b is 0, 1 or 2; R⁶ is selected from the group consisting of: halo, (C₁-C₆)alkyl, cyano, or (C₁-C₆)alkylcarbonyl; c is 1; and R⁷ is selected from the group consisting of: (C₁-C₆)alkylsulfonylaminocarbonyl(C₁-C₆)alkoxy, (C₂-C₉)heteroarylaminocarbonyl(C₁-C₆)alkoxy, (C₁-C₆)alkylsulfonylaminocarbonyl, aminocarbonyl, aminosulfonyl or carboxy.

The most preferred compounds of the formula I are those selected from the group consisting of:

2-(4-Chloro-phenoxy)-1-(4-phenoxy-piperidin-1-yl)-ethanone;

2-(4-Chloro-phenoxy)-1-[4-(4-fluoro-phenoxy)-piperidin-1-yl]-ethanone;

5-Chloro-2-{2-[4-(4-fluoro-phenoxy)-piperidin-1-yl]-2-oxo-ethoxy}-benzamide;

(5-Chloro-2-{2-[4-(4-fluoro-phenoxy)-piperidin-1-yl]-2-oxo-ethoxy}-phenyl)-urea; 5-Chloro-2-{(2,4-cis)-(2,5-trans)-2-[4-(4-fluoro-phenoxy)-2,5-dimethyl-piperidin-1-yl]-2-oxo-ethoxy}-benzamide;

(2,4-cis)-(2,5-trans)-5-Chloro-2-{2-[4-(4-fluoro-phenoxy)-2,5-dimethyl-piperidin-1-yl]-2-oxo-ethoxy}-phenyl)-acetic acid;

N-[(5-Chloro-2-{(2,4-cis)-(2,5-trans)-2-[4-(4-fluoro-phenoxy)-2,5-dimethyl-piperidin-1-yl]-2-oxo-ethoxy}-phenyl)-acetyl]-methanesulfonamide;

2-(5-Chloro-2-{2-[(2,4-cis)-(2,5-trans)-4-(4-fluoro-phenoxy)-2,5-dimethyl-piperidin-1-yl]-2-oxo-ethoxy}-phenyl)-acetamide;

(5-Chloro-2-{2-[4-(4-fluoro-phenoxy)-piperidin-1-yl]-2-oxo-ethoxy}-phenyl)-acetic acid;

N-[(5-Chloro-2-{2-[4-(4-fluoro-phenoxy)-piperidin-1-yl]-2-oxo-ethoxy}-phenyl)-acetyl]-methanesulfonamide; and

5-Chloro-2-{2-[(2,4-cis)-(2,5-trans)-4-(4-fluoro-phenoxy)-2,5-dimethyl-piperidin-1-yl]-2-oxo-ethoxy}-benzamide.

The present invention also relates to a pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. This invention also relates to a pharmaceutical composition for treating or preventing cancer metastasis, including but not limited to breast cancer. This invention also relates to a pharmaceutical composition for preventing production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This invention also relates to a pharmaceutical composition for preventing tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The present invention also relates to a pharmaceutical composition for treating or preventing a disorder or condition that can be treated or prevented by inhibiting chemokine binding to the receptor CCR1 in a mammal, preferably a human, comprising an amount of a compound of the formula I, or a pharmaceutically acceptable salt thereof, effective in treating or preventing such disorder or condition and a pharmaceutically acceptable carrier. Examples of such disorders and conditions are those enumerated in the preceding paragraph.

The present invention also relates to a method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. The present invention also relates to a method for treating or preventing cancer metastasis, including but not limited to breast cancer. The present invention also relates to a method for preventing the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith).

The present invention also relates to a method for preventing tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The present invention also relates to a method for treating or preventing a disorder or condition that can be treated or prevented by antagonizing the CCR1 receptor in a mammal, preferably a human, comprising administering to a mammal in need of such treatment or prevention an amount of a compound of the formula I, or a pharmaceutically acceptable salt thereof, that is effective in treating or preventing such disorder or condition.

The present invention also relates to a pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; and sequelae associated with certain cancers such as multiple myeloma. Pharmaceutical compositions of this invention are also potentially useful for the treatment or prevention of cancer metastasis, including but not limited to breast cancer. Pharmaceutical compositions of this invention may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Pharmaceutical compositions of this invention may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviroses, Herpes viruses (Herpes zoster and Herpes simplex) fungal meningitis, lyme disease, malaria) in a mammal, preferably a human, comprising a CCR1 receptor antagonizing effective amount of a compound of the formula I, or a pharmaceutically acceptable salt thereof, and a pharmaceutically acceptable carrier.

The present invention also relates to a pharmaceutical composition for treating or preventing a disorder or condition that can be treated or prevented by antagonizing the CCR1 receptor in a mammal, preferably a human, comprising a CCR1 receptor antagonizing effective amount of a compound of the formula I, or a pharmaceutically acceptable salt thereof, and a pharmaceutically acceptable carrier.

The present invention also relates to a method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, preferably a human, comprising administering to a mammal in need of such treatment or prevention a CCR1 receptor antagonizing effective amount of a compound of formula I, or a pharmaceutically acceptable salt thereof.

### DETAILED DESCRIPTION OF THE INVENTION

### Preparation A

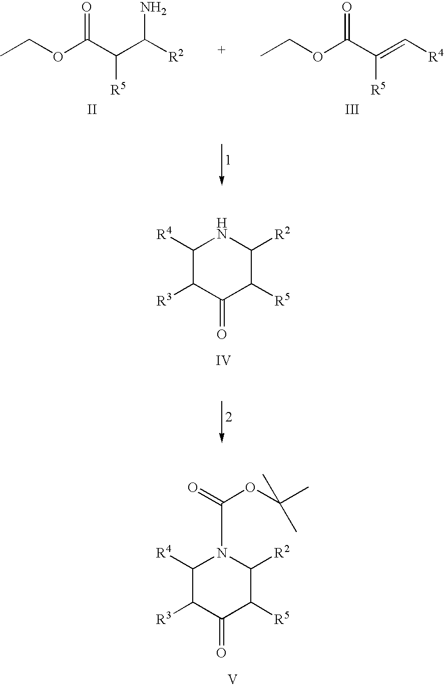

### Preparation B

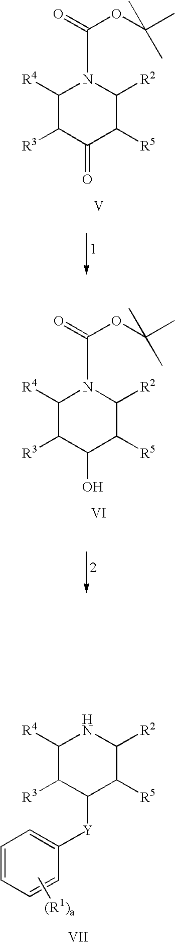

### Preparation C

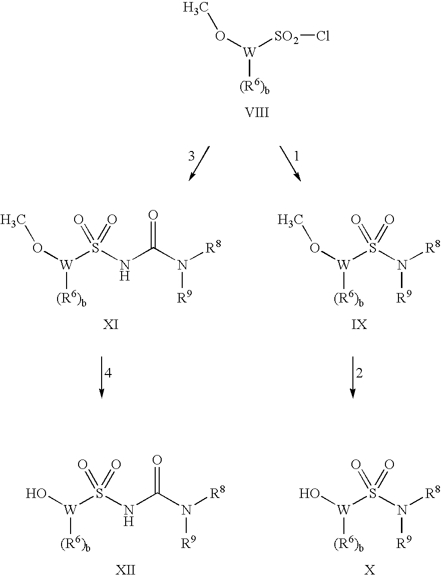

### Preparation D

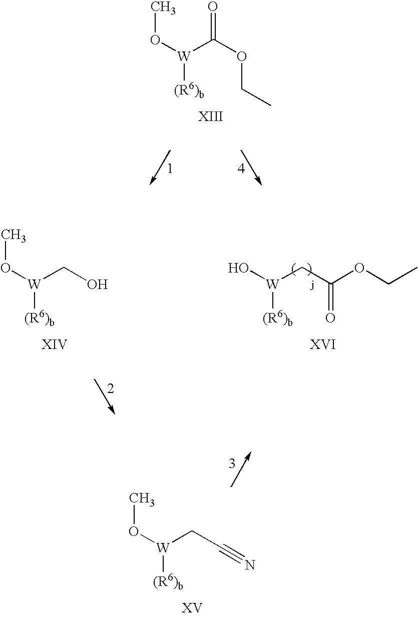

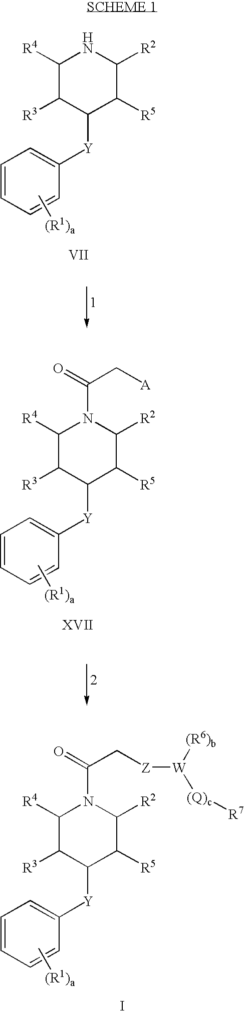

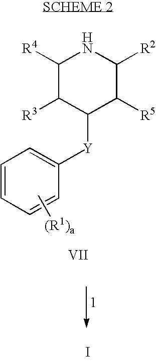

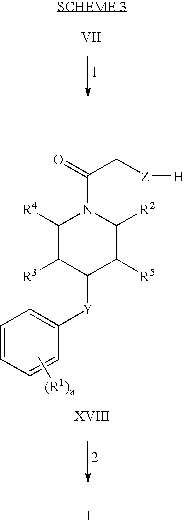

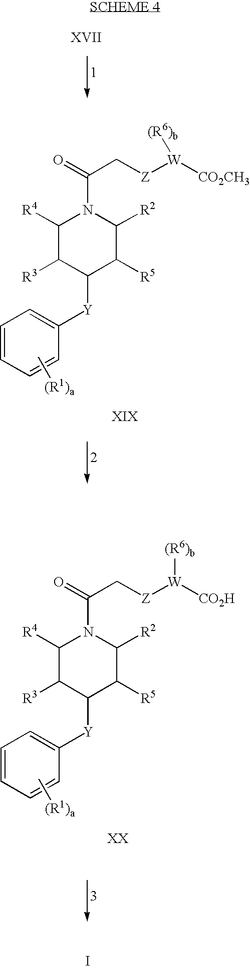

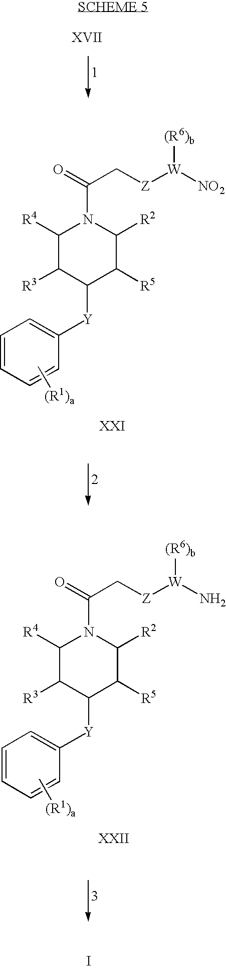

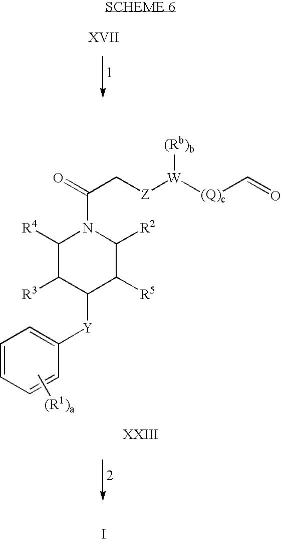

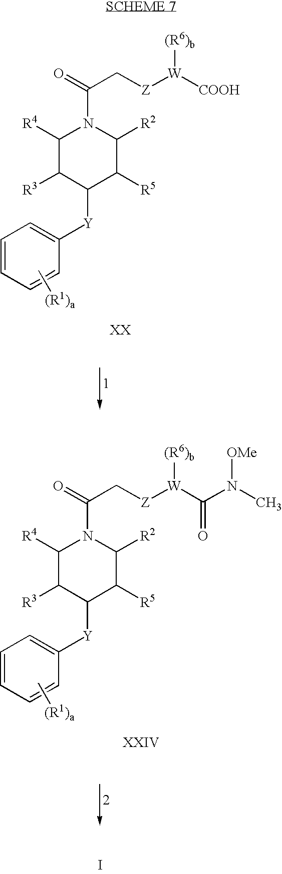

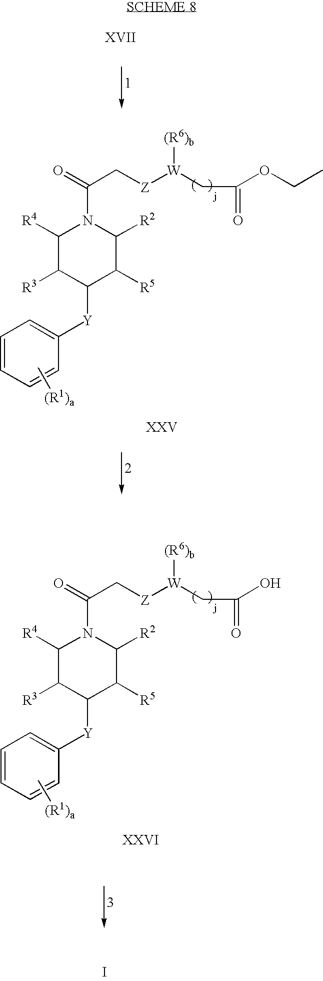

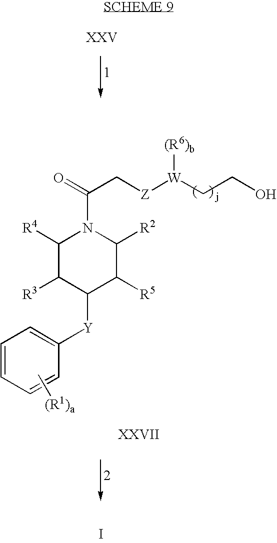

In reaction 1 of Preparation A, the compound of formula II is converted to the corresponding compound of formula IV by treating II with a compound of formula III in the presence of a base, such as sodium methoxide and heat.

In reaction 2 of Preparation A, the compound of formula IV is converted to the corresponding compound of formula V by reacting with carbonic acid di-tert-butyl ester in the presence of a base, such as sodium hydroxide, at ambient temperature for a time period between 5 hours and 15 hours, preferably around 12 hours.

In reaction 1 of Preparation B the compound of formula V, which is either commercially available or has been prepared according to Preparations A, is converted to the corresponding compound of formula VI by reacting with a reducing agent, such as L-selectride, in an aprotic solvent, such as tetrahydrofuran, to give a diastereomeric mixture of alcohols, which are separated at this stage by silica gel chromatography.

In reaction 2 of Preparation B the desired alcohol is then converted to the corresponding compound of formula VII by treating the alcohol VI with triphenyl phosphine and diethyl azodicarboxylate in the presence of a nucleophile of the formula:

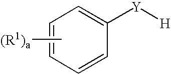

where in Y is oxygen and a is 1, 2, 3, 4 or 5. Finally, the resulting BOC protecting group on the arylether is removed with trifluoro acetic acid in an aprotic solvent, such as methylene chloride, to give the corresponding compound of formula VII. In the case that Y is NH, a compound of formula V is treated with a compound of the formula:

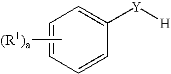

wherein Y is NH and a is 1, 2, 3, 4, or 5, in the presence of a reducing agent, such as sodium cyanoborohydride, in the presence of a polar aprotic solvent, such as dichloroethane. Deprotection with trifluoroacetic acid give the corresponding compound of formula VII.

In reaction 1 of the Preparation C, the compound of formula VIII is converted to the corresponding compound of formula IX by reacting VIII with an appropriate amine of the formula, HNR⁸R⁹, wherein R⁸ and R⁹ are each independently selected from a group, including but not limited to, hydrogen, a nitrogen containing (C₂-C₉)heterocycloalkyl or (C₂-C₉)heteroaryl group or an optionally substituted (C₁-C₆)alkyl, or R¹⁸ and R₁₉ are taken together with the nitrogen to which they are attached to form (C₂-C₉)heterocycloalkyl or (C₂-C₉)heteroaryl group, in the presence of a polar aprotic solvent, such as methylene chloride. The reaction mixture is stirred, at ambient temperature, for a time period between about 1 hour to about 24 hours, preferably about 12 hours.

In reaction 2 of Preparation C, the compound of formula IX is converted to the corresponding compound of formula X by reacting IX with thiophenol in the presence of a base, such as sodium hydride, and a polar aprotic solvent, such as dimethylformamide. The reaction is heated to reflux for a time period between about 1 hour to about 10 hours, preferably about 4 hours.

In reaction 3 of Preparation C, the compound of formula VIII is converted to the corresponding compound of formula XI by reacting VIII with sodium cyanate in the presence of pyridine and a polar aprotic solvent, such as acetonitrile. The reaction is stirred, at ambient temperature, for a time period between about 2 hours to about 18 hours, preferably about 10 hours. An appropriate amine of the formula HNR⁸R⁹, wherein R⁸ and R⁹ are each independently selected from a group, including but not limited to, hydrogen, a nitrogen containing (C₂-C₉)heterocycloalkyl or (C₂-C₉)heteroaryl group, or an optionally substituted (C₁-C₆)alkyl, or R¹⁸ and R₁₉ are taken together with the nitrogen to which they are attached to form (C₂-C₉)heterocycloalkyl or (C₂-C₉)heteroaryl group, is then added and the reaction mixture so formed is stirred, at ambient temperature, for a time period between about 2 hours to about 24 hours, preferably about 8 hours.

In reaction 4 of Preparation C, the compound of formula XI is converted to the corresponding compound of formula XII according to the procedure described above in reaction 2 of Preparation C.

In reaction 1 of Preparation D the compound of formula XIII is converted to the corresponding compound of the formula XIV by treating with a reducing agent, such as lithium aluminum hydride, in an aprotic solvent, such as tetrahydrofuran. The reaction mixture is heated to reflux for a time period between 1 hour and 6 hours, preferably about 2 hours.

In reaction 2 of Preparation D the compound of formula XIV is converted to the corresponding compound of the formula XV by first treating with an activating agent such as sulfonyl chloride, in the presence of an aprotic solvent, such as chloroform. The reaction is heated to reflux, for a time period between about 1 hour to about 10 hours, preferably about 3 hours. The resulting alkyl chloride is then treated with a cyanide source, such as potassium cyanide, in the presence of an aprotic solvent, such as acetonitrile. The reaction mixture is stirred at ambient temperature for a time period between about 1 hour to about 10 hours, preferably about 3 hours.

In reaction 3 of Preparation D the compound of formula XV is converted to the compound of formula XVI, wherein j is 1, by first treating XV with base, such as potassium hydroxide in water. The reaction mixture is heated to reflux for a time period between about 1 hour to about 10 hours, preferably about 6 hours. The resulting carboxylic acid is treated with acid, such as 47% aqueous hydrogen bromide to produce the deprotected phenol. The reaction mixture is heated to reflux for a time period between about 10 hours to about 30 hours, preferably about 24 hours. The deprotected phenol is finally converted to the corresponding compound of formula XVI, wherein j is 1, by refluxing in ethanol in the presence of an acid, such as sulfuric acid, for a time period between about 8 hours to about 16 hours, preferably about 12 hours.

In reaction 4 of Preparation D the compound of formula XIII is converted to the corresponding compound of formula XVI, wherein j is 2 or 3, by first treating the ester with a reducing agent, such as diisobutylaluminum hydride, in the presence of an aprotic solvent, such as toluene. The resulting aldehyde is treated with a phosphonium ylide derived from the phosphonium salt of the formula

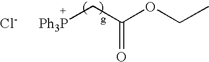

wherein g is 1 or 2, in the presence of an aprotic solvent, such as tetrahydrofuran. The reaction is refluxed for a time period between about 4 hours to about 16 hours, preferably about 10 hours. The resulting olefin is then reduced by shaking under a positive pressure of hydrogen in the presence of a catalyst, such as 20% palladium hydroxide on carbon, in the presence of a protic solvent such as ethanol. The methyl ether is deprotected according to the procedure described for reaction 3 of Preparation D.

In reaction 1 of Scheme 1, the compound of formula VII is converted to the corresponding compound of formula XVII by reacting VII with a compound of the formula, A—(C═O)—(CH₂)—A, wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 2 of Scheme 1, the compound of formula XVII is converted to the corresponding compound of formula I by reacting XVII with a compound of the formula

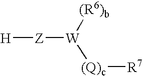

wherein Z is oxygen, which is either commercially available or is prepared according to Preparations D and E, in the presence of potassium carbonate, potassium iodide and an aprotic solvent, such as butanone. The reaction is heated to reflux for a time period between about 4 hours to about 8 hours, preferably about 6 hours.

In reaction 1 of Scheme 2, the compound of formula VII is converted to the corresponding compound of formula I by reacting VII with a compound of the formula

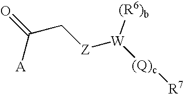

wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 1 of Scheme 3, the compound of formula VII is converted to the corresponding compound of formula XVIII by reacting VII with an carboxylic acid of the formula:

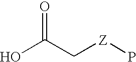

wherein Z—P is O—(C═O)—CH₃ or —NH—(C═O)—O-tBu, in the presence 4-dimethylaminopyridine, 1-(3-dimethylaminopropyl)-3-ethylcarbodiimine and a polar aprotic solvent, such as methylene chloride. In the case when Z—P is O—(C═O)—CH₃ then the resulting acetate is treated with base such as lithium hydroxide in a protic solvent such as a mixture of tetrahydrofuran, water and methanol, to give a compound of the formula XVIII. In the case when Z is —NH—(C═O)—O-tBu, the resulting amide is treated with an acid, such as trifluoroacetic acid, in an aprotic solvent, such as dichloromethane to give the compound of the formula XVIII.

In reaction 2 of Scheme 3, the compound of formula XVIII wherein Z is oxygen, or NH, is converted to the corresponding compound of formula I where W is a (C₂-C₉)heteroaryl group, by reacting with a compound of formula Hal-W, wherein Hal is a chloro or bromo and W is an appropriately functionalized heteroaryl group, in the presence of a base, such as sodium hydride, in an aprotic solvent, such as tetrahydrofuran.

In reaction 1 of Scheme 4, the compound of formula XVII is converted to the corresponding compound of formula XIX according to the procedure described above in reaction 2 of Scheme 1.

In reaction 2 of Scheme 4, the compound of formula XIX is converted to the corresponding compound of formula XX by reacting XIX with lithium hydroxide monohydrate in the presence of methanol, tetrahydrofuran and water. The reaction mixture is stirred overnight at ambient temperature.

In reaction 3 of Scheme 4, the compound of formula XX is converted to the corresponding amide or acylsulfonamide of formula I, by reacting XX with an appropriate amine or sulfonamide in the presence of 4-dimethylaminopyridine, 1-(3-dimethylaminopropyl)-3-ethylcarbodiimine and a polar aprotic solvent, such as methylene chloride. The resulting reaction mixture is stirred overnight at ambient temperature.

In reaction 1 of Scheme 5, the compound of formula XVII is converted to the corresponding compound of formula XXI according to the procedure described above in reaction 2 of Scheme 1.

In reaction 2 of Scheme 5, the compound of formula XXI is converted to the corresponding compound of formula XXII by hydrogenating XXI in the presence of a catalyst, such as platinum on carbon, and a polar protic solvent, such as ethanol. The reaction is carried out under a positive pressure of hydrogen gas between about 30 psi to about 40 psi, preferably about 35 psi, for a time period between about 15 minutes to about 1 hour, preferably 30 minutes.

In reaction 3 of Scheme 5, the compound of formula XXII is converted to the corresponding urea of formula I, by first reacting XXII with 4-nitrophenyl chloroformate in the presence of a base, such as pyridine, and a polar aprotic solvent, such as methlyene chloride, followed by reacting the intermediate so formed with an appropriate amine. The reaction mixture, so formed, is allowed to stir overnight at ambient temperature. To form the sulfonamides of formula I, the compound of formula XXII is reacted with an appropriate sulfonyl chloride in the presence of a base, such as triethylamine, and a polar aprotic solvent, such as methylene chloride. The reaction is stirred overnight at ambient temperature. To prepare cyanoguanidines of the formula I, the compound of formula XXII is first treated with sodium hydride in an aprotic solvent, such as tetrahydrofuran, followed by reacting the intermediate so formed with dimethyl-N-cyanodithio iminocarbonate. The resulting reaction mixture is heated to reflux overnight. The N-cyano-S-methyl-isothiourea intermediate is then reacted with an appropriate amine in the presence of a polar protic solvent, such as methanol, to form the cyanoguanidine of formula I. For the preparation of amides or the formula I, the compound of formula XXII is reacted with an appropriate acid in the presence of N-methylmorpholine, O-benzotriazole-1-yl-N,N,N,N-tetramethyluronium hexafluorophosphate and a polar aprotic solvent, such as methylene chloride, to form the amide of formula I. For secondary amine formation the compound of formula XXII is reacted with an appropriate aldehyde in the presence of a reducing agent, such as sodium triacetoxyborohydride, in the presence of a polar solvent, such as methanol.

In reaction 1 of Scheme 6, the compound of formula XVII is converted to the corresponding compound of formula XXIII, according to the procedure described above in reaction 2 of Scheme 1.

In reaction 2 of Scheme 6, the compound of formula XXIII is converted to the corresponding compound of formula I by reacting XXIII with an appropriate amine in the presence of a 10:1 ratio solution of dichloroethane/acetic acid. The reaction mixture is stirred, at ambient temperature, for a time period between about 30 minutes to about 2 hours, preferably about 1 hour. A reducing agent, such as sodium cyanoborohydride is than added to the mixture and the reaction is allowed to stir overnight at ambient temperature. If the amine thus formed is secondary, the compound of formula I may further be reacted according to the procedure described above in reaction 3 of Scheme 5, to provide ureas, sulfonamides, cyanoguanidines, or amides.

In reaction 1 of Scheme 7, the acid compound of formula XX is converted to the corresponding compound of formula XXIV by treating XX with thionyl chloride neat or in an aprotic solvent, at ambient temperature, for a time period between about 1 hour to about 24 hours, preferably 1 hour. The acid chloride so formed is dissolved in a polar aprotic solvent with a compound of the formula, (H₃CO)(H₃C)NH.HCl, in the presence of an amine base, such as triethylamine. The reaction mixture is stirred, at ambient temperature, for a time period between about 1 hour to about 48 hours, preferably about 12 hours.

In reaction 2 of Scheme 7, the amide compound of formula XXIV is converted to the corresponding compound of formula I by reacting XXIV with a (C₂-C₉)heteroaryl lithium reagent in the presence of a polar aprotic solvent at a temperature between about −100° C. to ambient temperature, preferably about −78° C. The resulting reaction mixture is stirred for a time period between about 1 hour to about 24 hours, preferably about 12 hours, at a temperature between about −78° C. to about 50° C., preferably about 20° C.

In reaction 1 of Scheme 8, the compound of formula XVII is converted to the corresponding compound of formula XXV, wherein j is 1, 2, or 3, according to the procedure described above in reaction 2 of Scheme 1.

In reaction 2 of Scheme 8, the compound of formula XXV, wherein j is 1, 2, or 3, is converted to the corresponding compound of formula XXVI, wherein j is 1, 2, or 3, according to the procedure described above in reaction 2 of Scheme 4.

In reaction 3 of Scheme 8 the compound of formula XXVI, wherein j is 1, 2, or 3, is converted to the corresponding amide or acylsulfonamide of the formula I, wherein j is 1, 2, or 3, by treating with an appropriate amine or sulfonamide according to the procedure described above in reaction 3 of Scheme 4. The compound of formula XXVI, wherein j is 1, 2, or 3, is converted to other compounds of formula I according to the procedures described above for Scheme 7.

In reaction 1 of Scheme 9 the compound of formula XXV, wherein j is 0, 1, 2, or 3, is converted to the corresponding compound of formula XXVII wherein j is 0, 1, 2, or 3, by reacting with a reducing agent, such as sodium borohydride, in a protic solvent, such as tert-butyl alcohol.

In reaction 2 of Scheme 9 the compound of formula XXVII, wherein j is 0, 1, 2, or 3, is converted to the corresponding compound of formula I by first treating with thionyl chloride, in the presence of an aprotic solvent, such as chloroform. The reaction is heated to reflux, for a time period between about 1 hour to about 10 hours, preferably about 3 hours. The resulting alkyl chloride is then treated with sodium sulfite in a polar protic solvent, such as ethanol and water, and heated to a temperature between 90° C. and 150° C., preferably around 110° C., for a time period between 10 and 20 hours, preferably 12 hours. To prepare sulfonamides or the formula I, the resulting sulfonate is treated with phosphorous pentachloride in an aprotic solvent, such as toluene, at a temperature between ambient and reflux, preferably at reflux for a time period between 1 hour and 8 hours, preferably 3 hours to give the corresponding sulfonyl chloride. The sulfonyl chloride is then reacted with an appropriate amine in a polar aprotic solvent, such as tetrahydrofuran, at ambient temperature for a time period between 3 hours and 24 hours, preferably 12 hours. The sulfonamide can be taken on further to acylsulfonamides of the formula I by treating with an acid chloride in the presence of base, such as triethylamine, in a aprotic solvent, such as dichloromethane, at ambient temperature.

Unless otherwise indicated, the pressure of each of the above reactions is not critical. Generally, the reactions are conducted at a pressure of about one to about three atmospheres, preferably at ambient pressure (about one atmosphere).

The compounds of the formula I that are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the basic compounds of this invention are readily prepared by treating the basic compound with a substantially equivalent amount of the chosen mineral or organic acid in an aqueous solvent medium or in a suitable organic solvent such as methanol or ethanol. Upon careful evaporation of the solvent, a solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

Those compounds of the formula I that are also acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The present invention also relates to compounds of formula I wherein any of the hydrogens may optionally be replaced by deuterium.

Unless otherwise indicated, the alkyl groups referred to herein may be linear or branched, and they may also be cyclic (e.g., cyclopropyl, cyclobutyl, cyclopentyl, cyclohexyl, cycloheptyl) or bicyclic (e.g., norbornanyl, bicyclo [3.2.1]octane) or contain cyclic groups. They may also contain zero to two levels of unsaturation and may be optionally substituted with 1 to 3 substituents independently selected from the group consisting of but not limited to: halo-, HO—, NC—, H₂N—, HO—(C═O)—.

Unless otherwise indicated, halogen includes fluorine, chlorine, bromine, and iodine.

(C₂-C₉)Heterocyclyl- when used herein refers to, but is not limited to, pyrrolidinyl, tetrahydrofuranyl, dihydrofuranyl, tetrahydropyranyl, pyranyl, thiopyranyl, aziridinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl and chromanyl. Said (C₂-C₉)heterocyclyl ring is attached through a carbon or a nitrogen atom.

(C₂-C₉)Heteroaryl when used herein refers to, but is not limited to, furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, and may be optionally substituted with 1 to 3 substituents independently selected from the group consisting of, but not limited to: H—, HO—, halo-, (C1-C8)alkyl- optionally substituted with 1-3 fluorine atoms, (C1-C8)alkyl-O— wherein the alkyl group is optionally substituted with 1-3 fluorine atoms, HO—(C1-C8)alkyl-, NC—, H₂N—, H₂N—(C1-C8)alkyl-, HO—(C═O)—, (C1-C8)alkyl-(C═O)—, (C1-C8)alkyl-(C═O)—(C1-C8)alkyl-, H₂N—(C═O)—, H₂N—(C═O)—(C1-C8)alkyl-, H₂NSO₂—, (C1-C8)alkyl-SO₂—NH—.

Aryl when used herein refers to phenyl or naphthyl which may be optionally substituted with 1 to 3 substituents independently selected from the group consisting of but not limited to: H—, HO—, halo-, (C1-C8)alkyl- optionally substituted with 1-3 fluorine atoms, (C1-C8)alkyl-O— wherein the alkyl group is optionally substituted with 1-3 fluorine atoms, HO—(C1-C8)alkyl-, NC—, H₂N—, H₂N—(C1-C8)alkyl-, HO—(C═O)—, (C1-C8)alkyl-(C═O)—, (C1-C8)alkyl-(C═O)—(C1-C8)alkyl-, H₂N—(C═O)—, H₂N—C═O)—(C1-C8)alkyl-, H₂NSO₂—, (C1-C8)alkyl-SO₂—NH—;

This invention also encompasses pharmaceutical compositions containing and methods of treating or preventing comprising administering prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain. This invention also provides for introduction of hydrogen isotopes (i.e., deuterium, tritium) by replacing ¹H₂ with ²H₂ or ³H₂ in the above procedure.

The compounds of this invention include all conformational isomers (e.g., cis and trans isomers. The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. In this regard, the invention includes both the E and Z configurations. The compounds of formula I may also exist as tautomers. This invention relates to the use of all such tautomers and mixtures thereof.

Compounds of the formula I and their pharmaceutically acceptable salts (hereinafter also referred to, collectively, as “the active compounds”) are potent inhibitors of MIP-1α (CCL3) binding to its receptor CCR1 found on inflammatory and immunomodulatory cells (preferably leukocytes and lymphocytes). The CCR1 receptor is also sometimes referred to as the CC-CKR1 receptor. These compounds also inhibit MIP-1α (and the related chemokines shown to interact with CCR1 (e.g., RANTES (CCL5), MCP-2 (CCL8), MCP-3 (CCL7), HCC-1 (CCL14) and HCC-2 (CCL15))) induced chemotaxis of THP-1 cells and human leukocytes and are potentially useful for the treatment and prevention of the following disorders and conditions: autoimmune diseases (such as rheumatoid arthritis, Takayasu arthritis, psoriatic arthritis, juvenile arthritis, ankylosing spondylitis, type I diabetes (recent onset), lupus, inflammatory bowel disease, Chrohn's disease, optic neuritis, psoriasis, neuroimmunologic disease (multiple sclerosis (MS) primary progressive MS, secondary progressive MS, chronic progressive MS, progressive relapsing MS, relapsing remitting MS, worsening MS), polymyalgia rheumatica, uveitis, thyroiditis and vasculitis); fibrosis (such as pulmonary fibrosis (for example idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic, dermatitis); acute and chronic inflammatory conditions including ocular inflammation, stenosis, lung inflammation (such as chronic bronchitis, chronic obstructive pulmonary disease, adult Respiratory Distress Syndrome, Respiratory Distress Syndrome of infancy, immune complex alveolitis), vascular inflammation resulting from tissue transplant or during restenosis (including, but not limited to, restenosis following angioplasty and/or stent insertion) and other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and chronic transplant rejection (including xeno-transplantation); HIV infectivity (co-receptor usage); granulomatous diseases (including sarcoidosis, leprosy and tuberculosis); Alzheimer's disease; chronic fatigue syndrome; pain; atherosclerosis; conditions associated with leptin production (such as obesity, cachexia, anorexia, type II diabetes, hyperlipidemia and hypergonadism); and sequelae associated with certain cancers such as multiple myeloma. This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention can be assessed according to procedures know to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, are placed into the lower chambers of the Boyden chamber. A polycarbonate filter is then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of>90% to be considered a valid assay.

All of the compounds of the invention illustrated in the following examples had IC₅₀ of less than 10 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use. The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents can be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

The compounds of the invention may also be utilized in combination therapy with other therapeutic agents such as those that inhibit immune cell activation and/or cytokine secretion or action (i.e. Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-Ig), BMS-224818 (CTLA4-Ig), RG-1046 (CTLA4-Ig), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, Anti-interleukin-6 receptor monoclonal antibody (MRA)), NSAIDS (aspirin, acetaminophen, naproxen, ibuprofen, ketoprofen, diclofenac and piroxicam), COX-2 inhibitors (Celecoxib, Valdecoxib, Rofecoxib, Parecoxib, Etoricoxib, L-745337, COX-189, BMS-347070, S-2474, JTE-522, CS-502, P-54, DFP), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

### EXAMPLES

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification.

### Example 1

### (+)-2-(5-Chloro-2-{(2,4-cis)-(2,5-trans)-2-[4-(4-fluoro-phenoxy)-2,5-dimethyl-piperidin-1-yl]-2-oxo-ethoxy}-phenyl)-acetamide

(5-Chloro-2-methoxy-phenyl)-methanol

To a solution of 5-chloro-2-methoxy-benzoic acid methyl ester (20 grams, 9.97 mmol) in THF (100 mL) at 0C was added dropwise a solution of lithium aluminum hydride (210 mL, 210 mmol, 1M soln. in THF). The solution was then warmed to reflux for 2 hours. The reaction was cooled to 0° C. and carefully quenched by the addition of cold water. The mixture was filtered through celite and the filter cake was washed with diethyl ether. The filtrate was washed with saturated aqueous sodium hydrogen carbonate then dried over magnesium sulfate. Concentration in vacuo gave the title compound (17.24 grams).

(5-Chloro-2-methoxy-phenyl)-acetonitrile

To a solution of (5-chloro-2-methoxy-phenyl)-methanol (17.1 grams, 99.06 mmol) in methylene chloride (100 mL) was added thionyl chloride (14.5 mL). The reaction was stirred at reflux for 3 hours, cooled to room temperature and concentrated in vacuo. The crude product was dissolved in methylene chloride and washed with saturated aqueous sodium hydrogen carbonate then dried over magnesium sulfate. Concentration in vacuo gave the benzyl chloride intermediate (18.43 grams). To a solution of the chloro compound in acetonitrile (100 mL) was added potassium cyanide (12.5 grams, 193 mmol) and 18-crown-6 (2.54 grams, 9.64 mmol). The reaction was stirred 12 hours at ambient temperature, diluted with ethyl acetate and washed with aqueous sodium hydrogen, carbonate. The organics were dried over magnesium sulfate and concentrated in vacuo. The crude product was purified by passing it through a pad of silica gel, eluting with methylene chloride, thus giving the title compound (17.2 grams).

(5-Chloro-2-methoxy-phenyl)-acetic Acid

To a solution of (5-chloro-2-methoxy-phenyl)-acetonitrile (17.2 grams, 96.3 mmol) in ethanol (200 mL) and water (20 mL) was added potassium hydroxide (27 grams, 481 mmol). The reaction was heated to reflux for 12 hours, cooled and concentrated in vacuo. The remaining solution was made acidic with aqueous hydrochloric acid (3 M) and extracted with diethyl ether. The organics were dried over magnesium sulfate and concentrated in vacuo to give the title compound (15.65 grams).

(5-Chloro-2-hydroxy-phenyl)-acetic Acid Ethyl Ester

A solution of (5-chloro-2-methoxy-phenyl)-acetic acid (15.54 grams, 77.5 mmol) in 48% aqueous hydrogen bromide was heated to reflux for 20 hours. The solution was cooled, diluted with water and extracted with diethyl ether. The organics were dried over magnesium sulfate and concentrated in vacuo. The crude product was purified by trituration in 2:1 methylene chloride:hexanes to give (5-chloro-2-hydroxy-phenyl)-acetic acid (12.78 grams). This was dissolved in a solution of ethanol saturated with hydrochloric acid and stirred 12 hours. The reaction was concentrated in vacuo, then the crude product was dissolved in diethyl ether and washed with saturated aqueous sodium hydrogen carbonate. The organics were dried over magnesium sulfate and concentrated in vacuo to give the title compound (12.7 grams).

(trans)-2,5-Dimethyl-piperidin-4-one

To a solution of 3-amino-butyric acid ethyl ester (20 ml, 149 mmol) in 2-propanol (8 ml) was added 2-methyl-acrylic acid methyl ester (17 ml, 159 mmol) and ammonium chloride (500 mg, 9.3 mmol). The reaction was refluxed for 4 hours, cooled and concentrated in vacuo to give 3-(2-methoxycarbonyl-propylamino)-butyric acid ethyl ester. The 3-(2-methoxycarbonyl-propylamino)-butyric acid ethyl ester was dissolved in toluene (100 ml) and heated to reflux. To this was added a 25% by weight solution of sodium methoxide in methanol (35 ml, 0.135 mmol) via drop funnel. The reaction was fitted with a condenser and methanol was azeotroped off at a vapor pressure of 100 to 110° C. After the methanol was azeotroped off, the reaction was heated at 110° C. for one hour. The reaction was then cooled to ambient temperature, treated with concentrated hydrochloric acid (50 ml), and refluxed for 3 hours. The reaction was cooled to ambient temperature and neutralized with solid sodium hydrogen carbonate. The reaction was cooled to 0° C. and saturated aqueous sodium hydroxide was then added until pH=11 was achieved. After stirring for one hour, the reaction was extracted with chloroform (3 times). The organic layers were combined, dried over magnesium sulfate, filtered and concentrated. The crude product was purified by vacuum distillation to give the title compound (3.68 grams, 21% yield).

(2,5-trans)-2,5-Dimethyl-4-oxo-piperidine-1-carboxylic Acid tert-Butyl Ester

To a solution of (trans)-2,5-dimethyl-piperidin-4-one (3.68 grams, 28.9 mmol) in tert-butyl alcohol (50 ml) and water (50 ml) was added sodium hydroxide (2.0 grams, 50 mmol) and di-tert-butyl-dicarbonate (7.0 grams, 32 mmol). The reaction was stirred at ambient temperature overnight. The reaction was diluted with water and extracted with diethyl ether (3 times). The organic layers were combined, dried over magnesium sulfate and concentrated to give the title compound (4.33 grams, 60% yield).

(2,4-trans)-(2,5-trans)-4-Hydroxy-2,5-dimethyl-piperidine-1-carboxylic Acid tert-Butyl Ester and (2,4-cis)-(2,5-trans)-4-Hydroxy-2,5-dimethyl-piperidine-1-carboxylic Acid tert-Butyl Ester

To a solution of (trans)-2,5-dimethyl-4-oxo-piperidine-1-carboxylic acid tert-butyl ester (2.08 grams, 9.15 mmol) in tetrahydrofuran (35 ml) at −78° C. under nitrogen was added L-selectride (15 ml, 15 mmol) via addition funnel. The reaction was stirred at −78° C. for 3 hours and then quenched with a phosphate buffer (pH=7). The reaction was extracted with ethyl acetate (2 times). The organic layers were combined, washed with brine, then dried over magnesium sulfate, filtered and concentrated. The crude product was purified by chromatography on silica gel to give the title compounds: (2,4-trans)-(2,5-trans) (1.1 grams, 52% yield) and (2,4-cis)-(2,5-trans) (2.41 grams, impure).

(2,4-cis)-(2,5-trans)-4-(4-Fluoro-phenoxy)-2,5-dimethyl-piperidine-1-carboxylic Acid tert-Butyl Ester

To a solution (2,4-cis)-(2,5-trans)-4-hydroxy-2,5-dimethyl-piperidine-1-carboxylic acid tert-butyl ester (1.1 grams, 4.79 mmol) in tetrahydrofuran (25 ml) was added triphenyl phosphine (1.91 grams, 7.28 mmol), 4-fluoro-phenol (865 mg, 7.7 mmol) and diethyl azidocarboxylate (1.2 ml, 7.6 mmol). The reaction was stirred overnight at ambient temperature. The reaction was then concentrated and purified by chromatography on silica gel giving the title compound (500 mg, 32% yield).

(2,4-cis)-(2,5-trans)-2-Chloro-1-[4-(4-fluoro-phenoxy)-2,5-dimethyl-piperidin-1-yl]-ethanone

To a solution of (2,4-cis)-(2,5-trans)-4-(4-fluoro-phenoxy)-2,5-dimethyl-piperidine-1-carboxylic acid tert-butyl ester (500 mg, 1,54 mmol) in dichloromethane (15 ml) was added trifluoroacetic acid (1.5 ml). The reaction was stirred at ambient temperature for 2 hours. The reaction was quenched with saturated aqueous sodium hydrogen carbonate and extracted with dichloromethane (2 times). The organic layers were combined, dried over magnesium sulfate, filtered and concentrated in vacuo. The resulting residue was dissolved in methylene chloride (10 ml), and treated with triethylamine (325 μL, 2.33 mmol) and chloroacetyl chloride (150 μL, 1.96 mmol). The reaction was stirred at ambient temperature for 3 hours, concentrated in vacuo, and purified by chromatography on silica gel to give the title compound (301 mg, 65% yield).

(2,4-cis)-(2,5-trans)-(5-Chloro-2-{2-[4-(4-fluoro-phenoxy)-2,5-dimethyl-piperidin-1-yl]-2-oxo-ethoxy}-phenyl)-acetic Acid Ethyl Ester

To a solution of (2,4-cis)-(2,5-trans)-2-chloro-1-[4-(4-fluoro-phenoxy)-2,5-dimethyl-piperidin-1-yl]-ethanone (150 mg, 0.50 mmol) in 2-butanone (1 ml) was added (5-chloro-2-hydroxy-phenyl)-acetic acid ethyl ester (125 mg, 0.58 mmol), potassium carbonate (175 mg, 1.26 mmol) and potassium iodide (85 mg, 0.512 mmol). The reaction was heated at 60° C. overnight. The reaction was cooled, diluted with water and extracted with ethyl acetate (2 times). The organic layers were combined, dried over magnesium sulfate, filtered and concentrated in vacuo. Chromatography on silica gel gave the title compound (174 mg, 73% yield).

(5-Chloro-2-{2,4-cis)-(2,5-trans)-2-[4-(4-fluoro-phenoxy)-2,5-dimethyl-piperidin-1-yl]-2-oxo-ethoxy}-phenyl)-acetic Acid

To a solution of (2,4-cis)-(2,5-trans)-(5-chloro-2-{2-[4-(4-fluoro-phenoxy)-2,5-dimethyl-piperidin-1-yl]-2-oxo-ethoxy}-phenyl)-acetic acid ethyl ester (170 mg, 0.355 mmol) in a solution of tetrahydrofuran (1 ml), methanol (1 ml) and water (0.5 ml) was added lithium hydroxide monohydrate (22 mg, 0.523 mmol). The reaction was stirred at ambient temperature for three hours. The reaction was diluted with ethyl acetate and washed with 0.2 M aqueous hydrochloric acid solution and brine. The organic layer was separated, dried over magnesium sulfate, filtered and concentrated in vacuo. The crude product was triterated in diethyl ether to give the title compound (163.3 mg, 100% yield).

(2,4-cis)-(2,5-trans)-2-(5-Chloro-2-{2-[4-(4-fluoro-phenoxy)-2,5-dimethyl-piperidin-1-yl]-2-oxo-ethoxy}-phenyl)-acetamide

To a solution of (2,4-cis)-(2,5-trans)-(5-chloro-2-{2-[4-(4-fluoro-phenoxy)-2,5-dimethyl-piperidin-1-yl]-2-oxo-ethoxy}-phenyl)-acetic acid (50.6 mg, 0.112 mmol) in dichloromethane (1 ml) was added thionyl chloride (11 μl, 0.15 mmol). The reaction was stirred at ambient temperature for 2 hours. The reaction is cooled to 0° C. and quenched with ammonium hydroxide (2 ml, 33%) and allowed to warm to ambient temperature over 3 hours. The reaction was diluted with water and extracted with dichloromethane (2 times). The organics were combined, dried over magnesium sulfate, filtered, concentrated in vacuo and triturated in diethyl ether to give the title compound (48.2 mg, 95% yield, LRMS M+H 449.2).

The title compounds for Example 2-4 were prepared by a method analogous to that described in Example 1.

| | LRMS | |
|---|---|---|
| Example | M + H | IUPAC name |
| 2 | 450.2 | (2,4-cis)-(2,5-trans)-(5-Chloro-2- |
| | | {2-[4-(4-fluoro-phenoxy)-2, 5- |
| | | dimethyl-piperidin-1-yl]-2-oxo- |
| | | ethoxy}-phenyl)-acetic acid |
| 3 | 527.3 | (2,4-cis)-(2,5-trans)-N-[(5-Chloro- |
| | | 2-{2-?-(4-fluoro-phenoxy)-2,5- |
| | | dimethyl-piperidin-1-yl]-2-oxo- |
| | | ethoxy}-phenyl)-acetyl]- |
| | | methanesulfonamide |
| 4 | 435.0 | 5-Chloro-2-{(2,4-cis)-(2,5-trans)- |
| | | 2-[4-(4-fluoro-phenoxy)-2,5- |
| | | dimethyl-piperidin-1-yl]-2-oxo- |
| | | ethoxy}-benzamide |

### Example 5

### (5-Chloro-2-{2-[4-(4-fluoro-phenoxy)-piperidin-1-yl]-2-oxo-ethoxy}-phenyl)-urea

4-Hydroxy-piperidine-1-carboxylic Acid tert-Butyl Ester

To a solution of sodium hydroxide (12.6 grams, 31.5 mmol)) in water (25 ml) was added tert-butyl alcohol (25 ml), piperidin-4-ol (2.04 grams, 20.17 mmol) and di-tert-butyl-dicarbonate (5.07 grams, 23.23 mmol). The reaction was stirred at ambient temperature overnight. The reaction was diluted with 0.2 M aqueous hydrochloric acid and extracted with ethyl acetate (2 times). The organic layers were combined, dried over magnesium sulfate, filtered and concentrated in vacuo to give the title compound (4.57 g, >100%).

4-(4-Fluoro-phenoxy)-piperidine-1-carboxylic Acid tert-Butyl Ester

To a solution of 4-hydroxy-piperidine-1-carboxylic acid tert-butyl ester (4 grams, 19.8 mmol) in tetrahydrofuran (80 ml) was added 4-fluoro phenol (2.62 grams, 23.3 mmol), triphenyl phosphine (6.25 grams, 23.3 mmol), and diethyl azidocarboxylate (3.8 ml, 24.1 mmol). The reaction was stirred at ambient temperature overnight. The reaction was diluted with dichloromethane and washed with 0.2M aqueous sodium hydroxide. The organic layer was separated, dried over magnesium sulfate and concentrated to give a yellow oil. Chromatography on silica gel gave the title compound (4.08 grams, 70% yield).

4-(4-Fluoro-phenoxy)-piperidine

To a solution of 4-(4-fluoro-phenoxy)-piperidine-1-carboxylic acid tert-butyl ester (2.04 grams, 6.91 mmol) in dichloromethane was added trifluoroacetic acid (3 ml). The reaction was stirred at ambient temperature for 2.5 hours. The reaction was concentrated, diluted with dichloromethane and washed with saturated aqueous sodium hydrogen carbonate. The organic layer was separated, dried over magnesium sulfate, filtered and concentrated to give the title compound (1.24 grams, 92% yield).

2-Chloro-1-[4-(4-fluoro-phenoxy)-piperidin-1-yl]-ethanone

To a solution of 4-(4-fluoro-phenoxy)-piperidine (1.24 grams, 6.36 mmol) in dichloromethane was added triethyl amine (1.2 ml, 8.6 mmol) and chloroacetyl chloride (0.54 ml, 7.0 mmol). The reaction was stirred at ambient temperature for 30 minutes. The reaction was concentrated in vacuo and purified by silica gel chromatography to give the title compound (1.22 grams, 71% yield).

2-(4-Chloro-2-nitro-phenoxy)-1-[4-(4-fluoro-phenoxy)-piperidin-1-yl]-ethanone

To a solution of 2-chloro-1-[4-(4-fluoro-phenoxy)-piperidin-1-yl]-ethanone (594 mg, 2.188 mmol) in 2-butanone (10 ml) was added 4-chloro-2-nitro-phenol (427 mg, 2.46 mmol), potassium carbonate (655 mg, 4.74 mmol) and potassium iodide (372 mg, 2.24 mmol). The reaction was refluxed overnight. The reaction was then cooled, concentrated in vacuo and purified by silica gel chromatography to give the title compound (699 mg, 78% yield).

2-(2-Amino-4-chloro-phenoxy)-1-[4-(4-fluoro-phenoxy)-piperidin-1-yl]-ethanone

To a solution of 2-(4-chloro-2-nitro-phenoxy)-1-[4-(4-fluoro-phenoxy)-piperidin-1-yl]-ethanone (699 mg, 1.71 mmol) in ethanol (50 ml) was added platinum on carbon (65 mg, 5% on carbon). The reaction was subject to 30 psi hydrogen gas overnight. The reaction mixture was filtered through a 0.54 μM filter and concentrated in vacuo to give the title compound (611 mg, 94% yield).

(5-Chloro-2-{2-[4-(4-fluoro-phenoxy)-piperidin-1-yl]-2-oxo-ethoxy}-phenyl)-urea

To a solution of 2-(2-amino-4-chloro-phenoxy)-1-[4-(4-fluoro-phenoxy)-piperidin-1-yl]-ethanone (65 mg, 0.171 mmol) in dichloromethane (1 ml) was added triethylamine (60 μl, 0.429 mmol) and phenylchloroformate (36 μl, 0.286 mmol). The reaction was stirred at ambient temperature for 4 hours. The reaction was then concentrated in vacuo, and the resulting residue dissolved in methanol (4 ml) followed by bubbling in ammonia gas for 10 minutes. The reaction was capped and stirred overnight at ambient temperature. The reaction was then concentrated in vacuo and purified by silica gel chromatography to give the title compound (53.1 mg, 73%, LRMS M+H=421.9).

The title compounds for Example 6-10 were prepared by a method analogous to that described in Example 5.

| | | | LCMS |
|---|---|---|---|
| Example | R³ | | M + H |
| 6 | H | 2-(4-Chloro-phenoxy)-1-(4- | 346.1 |
| | | phenoxy-piperidin-1-yl)- | |
| | | ethanone | |
| 7 | F | 2-(4-Chloro-phenoxy)-1-[4- | 364.1 |
| | | (4-fluoro-phenoxy)- | |
| | | piperidin-1-yl]-ethanone | |
| 8 | F | 5-Chloro-2-{2-[4-(4-fluoro- | 406.8 |
| | | phenoxy)-piperidin-1-yl]-2- | |
| | | oxo-ethoxy}-benzamide | |
| 9 | F | N-[(5-Chloro-2-{2-[4-(4- | 499.1 |
| | | fluoro-phenoxy)-piperidin-1- | |
| | | yl]-2-oxo-ethoxy}-phenyl)- | |
| | | acetyl]- | |
| | | methanesulfonamide | |

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of the formula 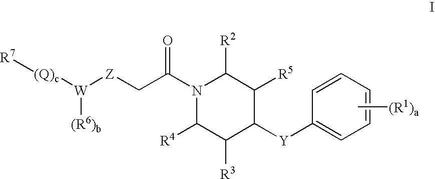
or pharmaceutically acceptable salts, tautomers, and pro-drugs thereof; wherein
a is 1, 2, 3, 4or 5;
b is 0, 1, 2, 3, or 4;
c is 0 or 1;
Q is (C₁-C₆)alkyl;
W is (C₆-C₁₀)aryl or (C₂-C₉)heteroaryl;
Y is oxygen, or NR⁸ wherein R⁸ is hydrogen or (C₁-C₆)alkyl;
Z is oxygen or NR⁹, where R⁹ is hydrogen, (C₁-C₆)alkyl, or acetyl;
each R¹ is independently selected from the group consisting of: hydrogen, halo, cyano, nitro, trifluoromethyl, trifluoromethoxy, (C₁-C₆)alkyl, hydroxy, (C₁-C₆)alkylcarbonyloxy, and (C₁-C₆)alkoxy;
R², R³, R⁴ and R⁵ are each independently hydrogen or (C₁-C₆)alkyl optionally substituted with 1 to 3 halo groups;
each R⁶ is independently selected from a list consisting of: hydrogen, halo, (C₁-C₆)alkyl optionally substituted with 1 to 3 halo groups; cyano, (C₁-C₆)alkoxy, aminocarbonyl, carboxy, (C₁-C₆)alkylcarbonyl, or (C₁-C₆)alkoxy optionally substituted by 1 to 3 halo groups; and
R⁷ is selected from a list consisting of hydrogen, halo, (C₁-C₆)alkyl optionally substituted with 1 to 3 halo groups, [(C₁-C₆)alkyl]₂amino(C₁-C₆)alkylaminocarbonyl, amino(C₁-C₆)alkylaminocarbonyl, (C₁-C₆)alkylamino(C₁-C₆)alkylaminocarbonyl cyano, (C₁-C₆)alkoxy, aminocarbonyl, (C₁-C₆)alkylaminocarbonyl, [(C₁-C₆)alkyl]₂aminocarbonyl, (C₁-C₆)alkylsulfonylamino, (C₁-C₆)alkylsulfonylaminocarbonyl, ureido, aminosulfonyl, [(C₁-C₆)alkyl]₂aminosulfonyl, (C₁-C₆)alkylaminosulfonyl, [(C₁-C₆)alkyl]₂aminocarbonyl(C₁-C₆)alkylaminocarbonyl, (C₁-C₆)alkylaminocarbonyl(C₁-C₆)alkylaminocarbonyl, aminocarbonyl(C₁-C₆)alkylaminocarbonyl, (C₁-C₆)alkylsulfonylamino, hydroxy(C₁-C₆)alkylcarbonylamino, ureido(C₁-C₆)alkylaminocarbonyl, [(C₁-C₆)alkyl]₂ureido(C₁-C₆)alkylaminocarbonyl, (C₁-C₆)alkylureido(C₁-C₆)alkylaminocarbonyl, (C₂-C₉)heteroarylaminocarbonyl, carboxy, (C₁-C₆)alkoxy(C₁-C₆)alkyl(C₂-C₉)heterocyclecarbonyl, (C₂-C₉)heterocyclecarbonyl, hydroxy(C₂-C₉)heterocyclecarbonyl, aminocarbonyl(C₂-C₉)heterocyclecarbonyl, carboxy(C₂-C₉)heterocyclecarbonyl, amino(C₂-C₉)heteroaryl(C₁-C₆)alkyl, (C₁-C₆)alkylamino(C₂-C₉)heteroaryl(C₁-C₆)alkyl, [(C₁-C₆)alkyl]₂amino(C₂-C₉)heteroaryl(C₁-C₆)alkyl, (C₂-C₉)heteroarylamino(C₁-C₆)alkyl, (C₂-C₉)heteroarylaminocarbonyl(C₁-C₆)alkoxy, (C₁-C₆)alkylsulfonylaminocarbonyl(C₁-C₆)alkoxy, aminocarbonyl(C₁-C₆)alkoxy, carboxy(C₁-C₆)alkoxy, aminosulfonyl, (C₁-C₆)alkylcarbonylaminosulfonyl, hydroxy(C₁-C₆)alkylcarbonylaminosulfonyl, (C₁-C₆)alkoxycarbonylaminosulfonyl, (C₁-C₆)alkoxy(C₁-C₆)alkylcarbonylaminosulfonyl, hydroxysulfonyl, hydroxy, hydroxy(C₁-C₆)alkylaminocarbonyl, carboxy(C₂-C₉)heterocycloxy or [carboxy][amino](C₁-C₆)alkoxy, aminocarbonyl(C₁-C₆)alkylcarbonylamino, (C₁-C₆)alkylaminocarbonyl(C₁-C₆)alkylcarbonylamino, [(C₁-C₆)alkyl]₂aminocarbonyl(C₁-C₆)alkylcarbonylamino, amino(C₁-C₆)alkylcarbonylamino, (C₁-C₆)alkylamino(C₁-C₆)alkylcarbonylamino, [(C₁-C₆)alkyl]₂amino(C₁-C₆)alkylcarbonylamino, ureido(C₁-C₆)alkylcarbonylamino, (C₁-C₆)alkylureido(C₁-C₆)alkylcarbonylamino, [(C₁-C₆)alkyl]₂ureido(C₁-C₆)alkylcarbonylamino, amino(C₁-C₆)alkylsulfonylamino, amino(C₁-C₆)alkylcarbonylaminosulfonyl, (C₁-C₆)alkylamino(C₁-C₆)alkylcarbonylaminosulfonyl, [(C₁-C₆)alkyl]₂amino(C₁-C₆)alkylcarbonylaminosulfonyl, aminosulfonylamino, (C₁-C₆)alkylaminosulfonylamino, [(C₁-C₆)alkyl]₂aminosulfonylamino, (C₂-C₉)heterocycloxy, (C₂-C₉)heteroaryloxy, (C₂-C₉)heterocycleamino, (C₂-C₉)heteroarylamino, amino(C₁-C₆)alkoxy, (C₁-C₆)alkylamino(C₁-C₆)alkoxy, [(C₁-C₆)alkyl]₂amino(C₁-C₆)alkoxy, amino(C₁-C₆)alkylamino, (C₁-C₆)alkylcarbonylamino(C₁-C₆)alkylamino, ureido(C₁-C₆)alkylamino, hydroxy(C₁-C₆)alkylamino, (C₁-C₆)alkoxy(C₁-C₆)alkylamino, and (C₁-C₆)alkylsulfonylamino(C₁-C₆)alkylamino;
with the proviso that at least one of R², R³, R⁴, and R⁵ is (C₁-C₆)alkyl.

2. A compound according to claim 1, wherein R¹ is halo; a is 1 or 2; Y is oxygen; Z is oxygen; W is phenyl; b is 0, 1 or 2 and R⁶ is selected from a list consisting of halo, (C₁-C₆)alkyl, cyano, and (C₁-C₆)alkylcarbonyl.

3. A compound according to claim 1, wherein R¹ is halo; a is 1 or 2; Y is oxygen; Z is oxygen or NH; W is pyridyl; b is 0, 1 or 2 and R⁶ is selected from a list consisting of halo, (C₁-C₆)alkyl, cyano, and (C₁-C₆)alkylcarbonyl.

4. A compound according to claim 1, wherein c is 0, and R⁷ is selected from a list consisting of (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, and carboxy(C₁-C₆)alkoxy.

5. A compound according to claim 1, wherein c is 1, and R⁷ is selected from a list consisting of (C₁-C₆)alkylsulfonylaminocarbonyl(C₁-C₆)alkoxy, (C₂-C₉)heteroarylaminocarbonyl(C₁-C₆)alkoxy, and (C₁-C₆)alkylsulfonylaminocarbonyl.

6. A compound according to claim 1, wherein R² and R³ are both methyl groups and R⁴ and R⁵ are both hydrogen.

7. A compound according to claim 2, wherein R² and R³ are methyl; R⁴ and R⁵ are hydrogen; R²and R³are trans; Y and R³ are trans; W is phenyl; c is 0; and R⁷ is selected from the group consisting of: (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, and carboxy(C₁-C₆)alkoxy.

8. A compound according to claim 3, wherein R² and R³ are methyl; R⁴and R⁵ are hydrogen; R²and R³ are trans; Y and R³ are trans; W is pyridyl; c is 0; and R⁷ is selected from the group consisting of: (C₁-C₆)alkylsulfonylamino, (C₁-C₆)alkylaminocarbonyl, aminosulfonyl, aminocarbonyl(C₁-C₆)alkylaminocarbonyl, (C₁-C₆)alkylaminocarbonyl, hydroxy(C₁-C₆)alkylcarbonylamino, aminocarbonylamino, carboxy(C₂-C₉)heterocycloalkoxy, carboxy(C₂-C₉)heteroarylcarbonyl, ureido(C₁-C₆)alkylaminocarbonyl, [(C₁-C₆)alkyl]₂amino(C₁-C₆)alkylaminocarbonyl, (C₁-C₆)alkylsulfonylaminocarbonyl(C₁-C₆)alkoxy, aminocarbonyl(C₁-C₆)alkoxy, and carboxy(C₁-C₆)alkoxy.

9. A compound according to claim 2, wherein R² and R³ are methyl; R⁴ and R⁵ are hydrogen; R² and R³ are trans; Y and R³ are trans; W is phenyl; c is 1; and R⁷ is selected from the group consisting of: (C₁-C₆)alkylsulfonylaminocarbonyl(C₁-C₆)alkoxy, (C₂-C₉)heteroarylaminocarbonyl(C₁-C₆)alkoxy, and (C₁-C₆)alkylsulfonylaminocarbonyl.

10. A compound according to claim 3, wherein R² and R³ are methyl; R⁴ and R⁵ are hydrogen; R² and R³ are trans; Y and R³ are trans; W is pyridyl; c is 1; and R⁷ is selected from the group consisting of: (C₁-C₆)alkylsulfonylaminocarbonyl(C₁-C₆)alkoxy, (C₂-C₉)heteroarylaminocarbonyl(C₁-C₆)alkoxy, and (C₁-C₆)alkylsulfonylaminocarbonyl.

11. A compound according to claim 1, wherein said compound is selected from the group consisting of:
2-(4-Chloro-phenoxy)-1-(4-phenoxy-piperidin-1-yl)-ethanone;
2-(4-Chloro-phenoxy)-1-[4-(4-fluoro-phenoxy)-piperidin-1-yl]-ethanone;
5-Chloro-2-{2-[4-(4-fluoro-phenoxy)-piperidin-1-yl]-2-oxo-ethoxy}-benzamide;
(5-Chloro-2-{2-[4-(4-fluoro-phenoxy)-piperidin-1-yl]-2-oxo-ethoxy}-phenyl)-urea;
5-Chloro-2-{(2,4-cis)-(2,5-trans)-2-[4-(4-fluoro-phenoxy)-2,5-dimethyl-piperidin-1-yl]-2-oxo-ethoxy}-benzamide;
(2,4-cis)-(2,5-trans)-5-Chloro-2-{2-[4-(4-fluoro-phenoxy)-2,5-dimethyl-piperidin-1-yl]-2-oxo-ethoxy}-phenyl)-acetic acid;
N-[(5-Chloro-2-{(2,4-cis)-(2,5-trans)-2-[4-(4-fluoro-phenoxy)-2,5-dimethyl-piperidin-1-yl]-2-oxo-ethoxy}-phenyl)-acetyl]-methanesulfonamide;
2-(5-Chloro-2-{2-[(2,4-cis)-(2,5-trans)-4-(4-fluoro-phenoxy)-2,5-dimethyl-piperidin-1-yl]-2-oxo-ethoxy}-phenyl)-acetamide;
(5-Chloro-2-{2-[4-(4-fluoro-phenoxy)-piperidin-1-yl]-2-oxo-ethoxy}-phenyl)-acetic acid;
N-[(5-Chloro-2-{2-[4-(4-fluoro-phenoxy)-piperidin-1-yl]-2-oxo-ethoxy}-phenyl)-acetyl]-methanesulfonamide; and
5-Chloro-2-{2-[(2,4-cis)-(2,5-trans)-4-(4-fluoro-phenoxy)-2,5-dimethyl-piperidin-1-yl]-2-oxo-ethoxy}-benzamide.

12. A pharmaceutical composition for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic-conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, comprising an amount of a compound according to claim 1, or a pharmaceutically acceptable salt thereof, that is effective in treating or preventing such disorder or condition and a pharmaceutically acceptable carrier.

13. A pharmaceutical composition for treating or preventing a disorder or condition that can be treated or prevented by inhibiting MIP-1αand/or RANTES binding to the receptor CCR1 in a mammal, comprising an amount of a compound according to claim 1, or a pharmaceutically acceptable salt thereof, effective in treating or preventing such disorder or condition and a pharmaceutically acceptable carrier.

14. A method for treating or preventing a disorder or condition selected from autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease; sequelae associated with certain cancers such as multiple myeloma; cancer metastasis, including but not limited to breast cancer; the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith); tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria) in a mammal, comprising administering to a mammal in need of such treatment or prevention an amount of a compound according to claim 1, or a pharmaceutically acceptable salt thereof, that is effective in treating or preventing such disorder or condition.

15. A method for treating or preventing a disorder or condition that can be treated or prevented by antagonizing the CCR1 receptor in a mammal, comprising administering to a mammal in need of such treatment or prevention an amount of a compound according to claim 1, or a pharmaceutically acceptable salt thereof, that is effective in treating or preventing such disorder or condition.
